(19) United States
(12) Patent Application Publication
(10) Pub. No.: US 2026/0096925 A1
(43) Pub. Date: Apr. 9, 2026

(54) Surgical System with Sensor Diagnostic Arrangement

(71) Applicant: JOHNSON & JOHNSON SURGICAL VISION, INC., Irvine, CA (US)

(72) Inventors: Vadim Gliner, Haifa (IL); Assaf Govari, Haifa (IL)

(21) Appl. No.: 18/905,311

(22) Filed: Oct. 3, 2024

(51) Int. Cl. A61F 9/007

(52) U.S. Cl. CPC A61F 9/00745

(57) ABSTRACT

A surgical system having a handpiece with a distal end; a pumping module configured for transmitting and/or evacuating fluid via the handpiece; a pressure sensor assembly positioned along fluid flow between the pumping module and distal end of the handpiece and configured for providing data on fluid pressure along the fluid flow; and a controller having one or more processors configured for operating the pumping module. The pressure sensor assembly includes at least three sensors positioned at two or more locations along the path of fluid flow between the pumping module and the distal end. The controller is configured for obtaining pressure data from the at least three sensors of the arrangement of sensors, and for processing the pressure data of the different sensors in accordance with a rotation rate of the pumping module to determine status of pressure data output by the at least three sensors.

### TECHNOLOGICAL FIELD

The present disclosure relates to surgical systems, and more particularly relates to phacoemulsification systems utilizing one or more pressure sensors.

### BACKGROUND

Phacoemulsification is a surgical procedure used to remove the natural lens tissue from the eye. This procedure may typically be used to treat cataracts, which are associated with clouding and hardening of the eye's lens and can cause blurred vision, difficulty seeing at night, and sensitivity to light. During the procedure, a surgeon makes a small incision in the patient’s cornea and inserts the distal end of a probe having a needle to break the cataract tissue into small pieces using ultrasound. The pieces of the lens are aspirated out of the eye and an artificial lens is inserted.

The operation may be performed using a phacoemulsification system comprising a handpiece, which comprises a hollow needle at a distal tip, a vibration mechanism (e.g. one or more piezoelectric crystals), irrigation and aspiration channels, and one or more sensors. The handpiece may be connected via a cable arrangement to a main unit providing one or more pumps for the aspiration channel, and pumping of irrigation fluid via an irrigation channel. Irrigation and aspiration channels extend through the cable arrangement to the handpiece, ending at the distal end of the handpiece to provide aspiration of fluids from the eye and providing the irrigation fluid into the eye.

In addition to aspiration of liquids and tissue parts from the eye, the phacoemulsification system operates to irrigate the eye, providing irrigation fluid that maintains intraocular pressure (IOP). Typically, the irrigation and aspiration rates are controlled by an automated process, while irrigation and/or aspiration pressures are monitored to maintain IOP.

### GENERAL DESCRIPTION

Monitoring irrigation and/or aspiration pressure during a phacoemulsification procedure is crucial to maintain IOP, and to avoid damage to the patient’s eye. While the phacoemulsification system typically comprises one or more pressure sensors, such pressure sensors may be faulty at times, or malfunction.

Malfunction of the pressure sensors may occur for various reasons, including repetitive autoclaving between procedures, impacts such as falling, manufacturing inaccuracies, or other factors. Such malfunction may compromise the safety of the procedure, affecting an operator/physician’s ability to monitor and control IOP. In some cases, the system can continue the procedure, utilizing pressure data obtained from one or more pressure sensors that are still operative. However, to maintain procedure safety, the malfunctioning sensors need to be identified.

The present disclosure provides a phacoemulsification system, and a method for operating a phacoemulsification system, utilizing an arrangement of sensors. The technique of the present disclosure utilizes pressure data obtained from different sensors of the pressure sensor assembly to determine data on status of the sensors and determine if one or more sensors malfunction.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 illustrates a surgical system according to some examples of the present disclosure;

FIG. 2 illustrated a handpiece of a surgical system and a pressure sensor assembly according to some examples of the present disclosure;

FIG. 3 illustrates data a processing path formed by a pressure sensor assembly and at least one processor according to some examples of the present disclosure; and

FIG. 4 illustrates a method according to some examples of the present disclosure.

### DETAILED DESCRIPTION OF EXAMPLES

In the following description, like elements are identified by the same numeral, and are differentiated, where required, by having a letter attached as a suffix to the numeral.

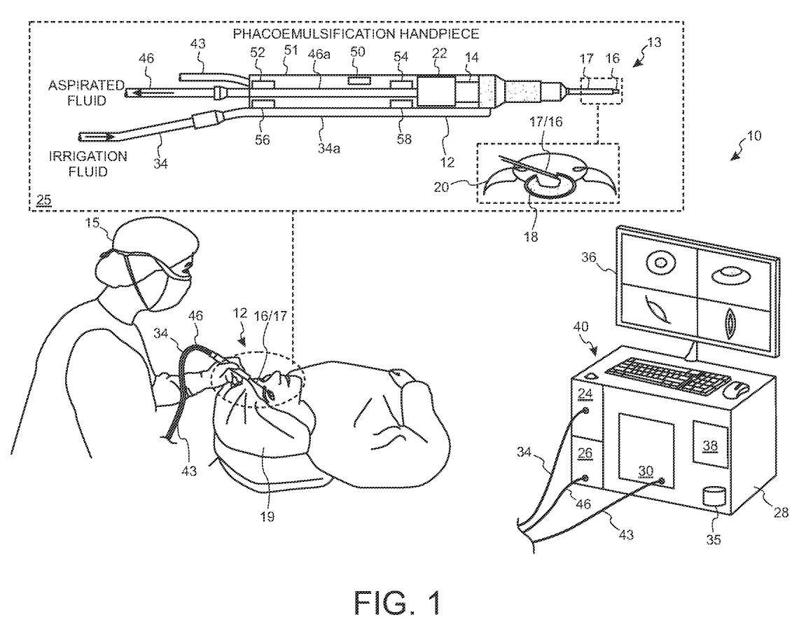

FIG. 1 is a pictorial view exemplifying a phacoemulsification system 10 including a phacoemulsification probe/handpiece 12 and a console 28 connected thereto via one or more cables (such as cables 34, 43, and 46). FIG. 1 includes an inset 25 illustrating additional details of the handpiece.

According to some examples of the present disclosure, phacoemulsification probe/handpiece 12 generally includes a hollow needle 16 and a coaxial irrigation sleeve 17. Irrigation sleeve 17 may be positioned to at least partially surround the needle 16, creating a fluid pathway between the external wall of the needle 16 and the internal wall of the sleeve 17. Needle 16 and sleeve 17 are also herein termed a needle-sleeve combination 13. Needle 16 is configured to be inserted into a capsular bag to emulsify lens 18 of an eye 20 of a patient 19. Needle 16 is shown in inset 25 as a straight needle, however, generally, any suitable shape of the needle 16 may be used with the phacoemulsification probe 12, for example, a curved or bent tip needle that is commercially available from Johnson & Johnson Surgical Vision, Inc., Irvine, CA, USA.

The physician 15, operating the system 10 may hold the handpiece 12 so as to perform a phacoemulsification procedure on the eye 20 of a patient 19. The physician 15 may activate the handpiece using a foot pedal (not shown in the figures) or any other suitable user interface, e.g., buttons, voice activated, etc. Handpiece 12 operates using a piezoelectric actuator 22 configured to vibrate horn 14 and needle 16 connected thereto, in one or more vibration modes of the combined horn and needle. The vibration of needle 16 is used to break up natural lens 18 into small pieces during the phacoemulsification procedure.

Phacoemulsification system 10 utilizes at least one controller 38 typically including at least one processor and corresponding memory units 35, typically associated with console 28. The at least one controller 38 is configured to execute selected computer readable codes including instructions for operating the phacoemulsification system 10 in one or more operations modes and in accordance with techniques described herein. It should be noted that certain actions associated with operation of the phacoemulsification system 10 are known in the art and are not specifically described herein. The operation of the at least one controller 38 include software and/or hardware functional modules relating to actions described in more detail below. One or more of the functions of the one or more processors of controller 38 may be carried out by suitable software stored in a memory 35. The software may be downloaded to a device in electronic format, over a network, for example. Alternatively, or additionally, the software may be stored in tangible, non-transitory computer-readable storage media, such as optical, magnetic, or electronic memory. Some or all of the functions of controller 38 may be combined in a single physical component, or, alternatively, implemented using multiple physical components. The physical components may comprise hard-wired or programmable devices, or a combination of the two.

Actuator 22 of the handpiece 12 is powered by a driving module 30 that may be placed within console 28. Module 30, under overall control of the controller 38, is configured to provide power to the actuator 22, via a microcontroller 50, e.g., located in the handpiece 12. Power for microcontroller 50, as well as control signals for the microcontroller, may be delivered to the microcontroller by a cable 43 from driving module 30.

During the phacoemulsification procedure, an irrigation pump 24, which may be located within console 28 or externally thereto, operates to pump irrigation fluid through an irrigation channel 34a in handpiece 12 and toward irrigation sleeve 17 so as to irrigate the eye. The fluid is pumped via an irrigation tubing line 34, running from the pump, which is coupled with channel 34a of the probe 12.

Additionally, an aspiration pump 26, which also may also be located within console 28 or externally thereto, operates to aspirate aspiration fluid, comprising fluid and waste matter (e.g., emulsified parts of the lens), from the patient’s eye 20 via needle 16, through an aspiration channel 46a in handpiece 12. Aspiration pump 26 produces a vacuum and is coupled with aspiration channel 46a by an aspiration tubing line 46.

The irrigation pump 24 and the aspiration pump 26 may be any pump known in the art (e.g., a peristaltic pump, a progressive cavity pump, or Venturi pump), and the pumps are under overall control of the controller 38. The irrigation and aspiration pumps operate to aspirate fluids from the patient’s eye, and irrigate the patient’s eye with irrigation fluid, to thereby maintain IOP and chamber stability. Generally, operation of the irrigation 24 and aspiration 26 pumps is controlled to maintain a desired pressure range within the patient’s eye to avoid damage to the structures of the eye.

To provide monitoring of pressure, the phacoemulsification system 100, and specifically the handpiece thereof, includes a pressure sensor assembly 51 including one or more sensors. The pressure sensor assembly 51 generally includes a selected number of sensors positioned for monitoring pressure variations along the aspiration and/or irrigation paths. In the exemplary configuration illustrated in FIG. 1, a first aspiration pressure sensor 52, also herein termed aspiration pressure sensor A, is coupled with aspiration channel 46a, in a proximal section of the channel, so as to couple with the aspiration fluid in the channel. A second aspiration pressure sensor 54, also herein termed aspiration pressure sensor B, is also coupled with aspiration channel 46a, in a distal section of the channel, so as to couple with the aspiration fluid in the channel. According to some examples of the present disclosure, the pressure sensor assembly 51 may include at least one additional aspiration sensor, positioned in the vicinity of aspiration sensor A, aspiration sensor B, or at a separate location. The aspiration pressure sensors are located at a known distance between each other, measured along aspiration channel 46a. Pressure variation between the sensors can thus be described in accordance with Bernoulli's principle of hydrodynamics.

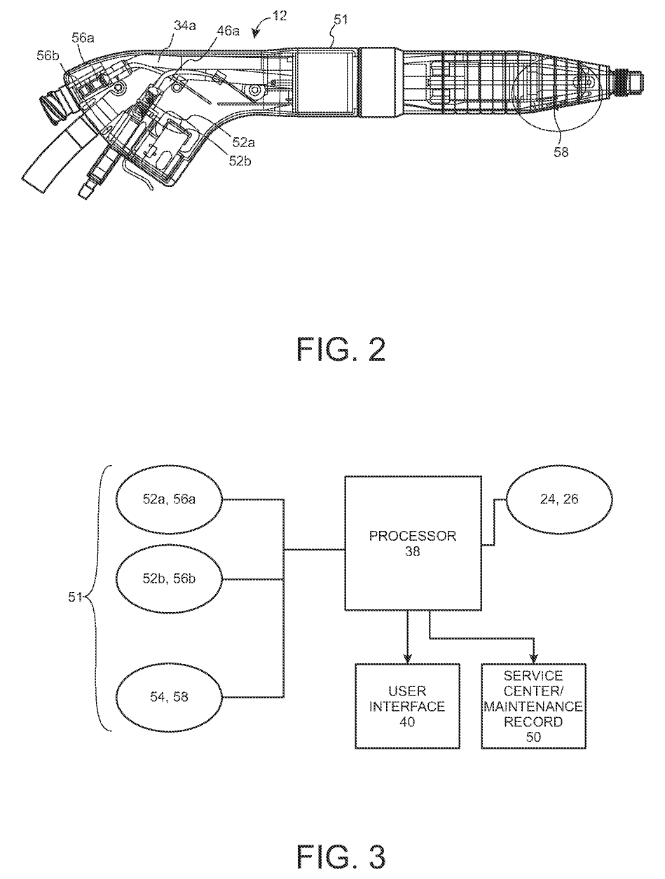

Similarly, the pressure sensor assembly 51 may include three or more irrigation sensors, as exemplified in FIG. 2. The first irrigation pressure sensor 56 illustrated in FIG. 1 can represent location of proximal pressure irrigation sensors 56a and 56b, which are directly exemplified in FIG. 2 coupled with irrigation channel 34a in a proximal section of the channel, and a third irrigation pressure sensor 58 also coupled with irrigation channel 34a, in a distal section of the channel. Further, as described above, the irrigation sensors may include at least one additional sensor located in the vicinity of irrigation pressure sensor 56 and/or of irrigation pressure sensor 58. The irrigation pressure sensors are located at a known distance between them, the distance being measured along irrigation channel 34a, providing that pressure variation between the sensor can be described in accordance with Bernoulli's principle of hydrodynamics.

The pressure sensor assembly 51, and the different aspiration and/or irrigation pressure sensors thereof, provide pressure output data to the at least one controller 38, using one or more communication lines and/or using wireless communication. For example, signals generated by the aspiration sensors, e.g., sensor 52 and sensor 54, and by the irrigation sensors, e.g., sensor 56 and sensor 58, are provided via cable 43, as illustrated in FIG. 1, to controller 38. The at least one controller 38 uses the pressure data collected from the pressure sensor assembly 51 to determine suitable aspiration and irrigation flow rates, and to provide suitable indication signals to the operator. Typically, irrigation pressure data is used to determine irrigation flow rate, and aspiration pressure data is used to determine aspiration flow rate. In some examples, the one or more processors of the at least one controller 38 utilize certain crosstalk between the irrigation and aspiration pressure data, to maintain proper IOP in the patient’s eye.

The apparatus illustrated in FIG. 1 may include various additional elements, which are omitted for clarity of presentation. For example, physician 15 typically performs the procedure using a stereomicroscope or magnifying glasses, neither of which are shown. Physician 15 may use other surgical tools, in addition to probe 12, which are also not shown to maintain clarity and simplicity.

The at least one controller 38 may perform additional processing on the pressure data, including one or more additional data, such as rotation rate of the aspiration and/or irrigation pumps, vibration mode and frequency of the actuator 22, etc. The at least one controller 38 may generate output data indicative of the processing, and provide output data through a corresponding user interface 40, e.g., using display 36. In an example, user interface 40 and display 36 may be one and the same, such as a touch screen graphical user interface.

The at least one controller 38 may receive user-based commands via a system user interface 40. Such user-based commands may include, but are not limited to, setting and/or adjusting a vibration mode and/or a frequency of piezoelectric actuator 22, setting and/or adjusting a stroke amplitude of needle 16, and setting and/or adjusting an irrigation flow rate and an aspiration flow rate of irrigation pump 24 and aspiration pump 26.

While physician 15 may set the irrigation flow rate and the aspiration flow rate to selected values, or may set the rates to respective default values, it will be understood that the actual rates of flow of both the irrigation fluid and the aspiration fluid may vary with time. The pumps themselves do not deliver the fluid at a constant rate, but rather vary the rate about a nominal set value in an oscillatory manner. In addition, flexing of the tubing connected to the pumps, as well as changes in the tubing terminations at the eye due to the procedure being performed, may cause the flow rate to change.

Generally, during a phacoemulsification procedure, irrigation fluid may flow through the irrigation channel and aspiration fluid may flow through the aspiration channel. In case of an occlusion, the fluid flow may stop for a certain period of time. The pressure registered by the aspiration and irrigation sensors, may vary from the IOP due to flow in the channel. In some examples, using the irrigation fluid pressure, IOP can be estimated based on the pressure in the irrigation channel. Accordingly, pressure data collected along the irrigation and aspiration channels can provide an operation/physician with valuable data on operation of the system as well as enable estimation on IOP of the patient. Thus, while not being directly equal, the pressure in the irrigation and/or aspiration channels provides an indication of the IOP, as well as an indication on variation in flow rate of the fluid being transferred. Accordingly, monitoring pressure data provides an important input to the physician 15 for safely performing the procedure.

While the phacoemulsification system 10 may preferably be manufactured of high-grade materials using high quality elements, the different pressure sensors may malfunction for various reasons. To enable monitoring of the pressure data and detection of malfunctioning sensors, the phacoemulsification system of the present disclosure utilizes a pressure sensor assembly 51 including three or more sensors along at least one of the irrigation and aspiration channels. Additionally, the at least one controller 38 is adapted for analyzing pressure data of the three or more sensors to determine if one or more of the sensors may be malfunctioning.

In this connection, reference is made to FIG. 2 exemplifying a probe/handpiece unit 12 of a phacoemulsification system according to some examples of the present disclosure. The handpiece 12 is generally configured as described above with reference to FIG. 1, while FIG. 2 specifically shows the arrangement of sensors including three or more sensors. As shown, irrigation channel 34a is associated with first proximal pressure sensor 56a and second proximal pressure sensor 56b and at least one distal pressure sensor 58. Additionally, in some examples, aspiration channel 46a may also be associated with first proximal pressure sensor 52a and second proximal pressure sensor 52b and at least one distal pressure sensor that is not specifically shown.

The three or more irrigation sensors 56a, 56b, and 58 collect pressure data indicative of pressure within the irrigation channel 34a, and transmit the pressure data to the at least one controller 38. Given a distance between the proximal sensors 56a and 56b, and the distal sensor 58, it is expected that these sensors will have certain pressure variations in accordance with Bernoulli's principle of hydrodynamics. The proximal pressure sensors 56a and 56b are located in close proximity to each other, and therefore are expected to provide pressure output with only small variations, and preferably equal pressure output. A generally similar pressure sensor assembly 51 may be associated with the aspiration channel 46a. This is illustrated in FIG. 2 by proximal pressure sensors 52a and 52b, while distal pressure sensor 54 is not directly illustrated here (shown in FIG. 1).

FIG. 2 illustrates the handpiece 12 as having two proximal pressure sensors 56a, 56b (or 52a, 52b) and a single distal pressure sensor 58. It should be noted that other sensor configurations may be used, including e.g., two distal pressure sensors and one or more proximal pressure sensors. Further, in some examples, the pressure sensor assembly 51 may include three or more sensors arranged in selected location along the irrigation channel 34a, where pressure variation between the sensors may be determined in accordance with the Bernoulli's principle based on location of the pressure sensors along path of the irrigation channel.

The at least one controller 38 may be configured to execute computer readable code, or be directly configured (e.g., by hardware configuration), to receive pressure data from the three or more pressure sensors associated with the irrigation (and/or aspiration) channel, and to process the pressure data to determine sensor status. In this connection reference is made to FIG. 3 exemplifying a signal communication path between the pressure sensor assembly 51 and the at least one controller 38 according to some examples of the present disclosure. As shown, three or more sensors, 52a, 52b, and 54 (or 56a, 56b, and 58) are configured to transmit, via communication wires or by wireless communication, pressure data to the at least one controller 38. The processor utilizes the pressure data obtained by the three or more sensors, and performs a sanity check processing to determine data on operation status of the three or more sensors. The at least one controller 38 is further configured to generate an output signal and transmit the output signal to the operator/physician via user interface 40. Additionally, in some examples the at least one processor may transmit operation status data to be stored in maintenance records 50 for further processing. The maintenance records 50 may be used for recording required maintenance actions such as replacing one or more malfunctioning sensors, calibrating sensors, etc.

The at least one controller 38 operates to perform a diagnostic routine for diagnosing operation status of the pressure sensors of the pressure sensor assembly 51. To In the diagnostic routine, the at least one controller 38 may operate to determine correlations between pressure data provided by the three or more sensors, and determine if any of the three or more sensors may be faulty. In this connection, the arrangement of sensors associated with the irrigation or aspiration channel may be marked as sensors A1, A2, and B, such that sensors A1 and A2 are two sensors located in close proximity (e.g., sensors 52a and 52b), and sensor B (e.g., sensor 54) is located at a selected distance from sensors A1 and A2. In this configuration, pressure data provided by sensors A1 and A2 is expected to match, i.e., relate to similar pressure values with minimal variations typically associated with accepted error margins. Further, sensor B is expected to provide pressure data that relates to pressure data of sensors A1 and A2, taking into account a distance between the sensors and variations associated with the Bernoulli principle. For example, pressure output of sensor B can typically be derived from pressure output of sensor A1 or A2 taking into account pressure drop associated with the Bernoulli principle. Further, in some examples, the at least one controller 38 may utilize additional data such as pump rotation rate or the irrigation 24 and/or aspiration 26 pumps, and determine correlation between pressure data and pump rotation rate. The correlation between pressure data and pump rotation rate may be based on pre-stored data stored in the memory 35.

For example, in a first scenario, sensors A1 and A2 transmit similar pressure data, and sensor B transmits pressure data having a variation that complies with the Bernoulli principle and distance between the sensors, to pressure data of sensors A1 and A2. In this scenario, the sensors are all marked as good. Further, as indicated above, variation of the pressure data from expected pressure in correlation with pump rotation rate may be used.

In a second possible scenario, sensors A1 and A2 provide different pressure readings, and pressure reading of sensors A2 and B match between them and optionally, correlate with rotation rate of the pump. In this scenario, sensor A1 is marked as faulty, while sensors A2 and B are marked as good.

In a third scenario, pressure data of sensors A1 and B match between them, and optionally correlate to pump rotation rate, and pressure data of sensor A2 does not match the pressure data of other sensors and/or pump rotation rate. In this scenario, the controller 38 may mark sensor A2 as faulty. Such marking may be associated with providing a signal to the operator via user interface 40 and/or generating a record in maintenance records 50.

In a fourth scenario, pressure readings of sensors A1 and A2 match between them, while not matching the pressure reading of sensor B. Readings of sensors A1 and A2 correlate with rotation rate of the pump. In this scenario, sensor B is marked as faulty, and sensors A1 and A2 are marked as good.

In accordance with correlations between pressure readouts of the three or more sensors, the at least one controller 38 generates an output signal indicating status of the sensors as being operating or faulty. The controller 38 transmits the output signal to at least one of the user interface 40 and maintenance record 50. In some examples, the controller 38 may further generate an output signal directed to a service center and/or generate an external notification indicating status of the sensors. Data on sensor operation status may be used by the physician for determining data quality and which sensors are to be used during operation. Additionally, the stored data may be used for later maintenance of the phacoemulsification system, indicating a need to replace the faulty sensors, or identify one or more other sources to the faulty readout.

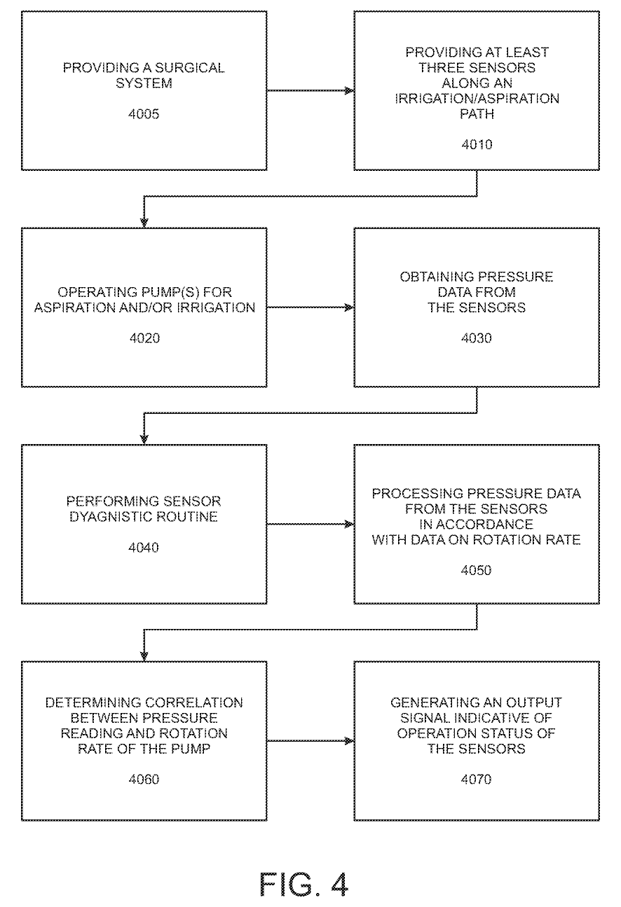

Reference is further made to FIG. 4 exemplifying a method according to some examples of the present disclosure. As shown, the method includes providing a surgical system (4005). The surgical system may include at least one pumping module, a pressure sensor assembly generally including at least three sensors (4010) positioned at two or more locations along a path of fluid flow between the at least one pumping module and a distal end of the handpiece. The at least one pumping module is configured for transmitting and/or evacuating fluid via the handpiece. The sensors of the sensor assembly may be placed at different locations along the irrigation and/or aspiration channel. For example, two or more sensors may be placed at a proximal location with respect to the distal end of the handpiece, and one or more additional sensors may be placed closer to the distal end. Alternatively, two or more sensors may be placed near the distal end of the handpiece, and one or more sensors may be placed at a proximal end of the handpiece. Further, in some examples, the three or more sensors may be placed at three or more different locations along the channel.

While the surgical system is being operated, the method includes operating the pumping module (4020) and further operating the pressure sensor assembly 51 for providing pressure reading data of the three or more sensors. Further, this also includes obtaining/collecting the pressure reading data from the three or more sensors (4030). The method further includes performing a sensor diagnostic routine (404) including processing the pressure data obtained from the sensors in accordance with data on rotation rate of the pump (4050) utilizes determining correlations between the pressure readings of the different (at least three) sensors (4060) to determine operation status of each sensor. To this end the method may utilize majority rule, correlation in accordance with Bernoulli's principle of hydrodynamics and distance between the sensors, and correlation with the rotation rate of the irrigation/aspiration pump. Following determining operation status of the three or more sensors, the method includes generating output data indicative of the operation status of each of the sensors (4070). The output data may indicate that the sensor status is “good” or “operating” on one hand, or “faulty” on the other hand. For example, output data may utilize display color of pressure readings, where pressure readings of operating sensors may be marked with one color, and pressure readings of faulty sensors may be marked in a different color. Further, the output signal may be used to avoid presentation of pressure readings of the faulty sensors.

### Examples

Example 1: A surgical system (10) comprising: a handpiece (12) having a distal end configured to be inserted into an eye (20) of a patient; a pumping module (24, 26) configured for transmitting and/or evacuating fluid via the handpiece (12); a pressure sensor assembly (51) positioned along a fluid flow path between the pumping module (24, 26) and the distal end of the handpiece (12) and configured for providing data on fluid pressure along the fluid flow path; and a controller (38) comprising one or more processors configured for operating the pumping module (24, 26); wherein the pressure sensor assembly (51) comprises at least three sensors (52a, 52b, 54, 56a, 56b, 58) positioned at two or more locations along the fluid flow path between the pumping module (24, 26) and the distal end; and wherein the controller (38) is configured to perform a sensor diagnostic routine by obtaining pressure data from the at least three sensors (52a, 52b, 54, 56a, 56b, 58), processing the pressure data from the at least three sensors (52a, 52b, 54, 56a, 56b, 58) in accordance with a rotation rate of the pumping module (24, 26), and determining malfunction status of the at least three sensors (52a, 52b, 54, 56a, 56b, 58).

Example 2: The surgical system (10) of example 1, wherein the sensor arrangement (51) comprises one or more sensors (52a, 52b, 54, 56a, 56b, 58) located within the handpiece (12).

Example 3: The surgical system (10) of example 1 or 2, wherein the three or more sensors (52a, 52b, 54, 56a, 56b, 58) are positioned in at least one of: at least two sensors are positioned at a distal end of the handpiece (12), and at least one sensor is located at a proximal end of the handpiece (12); or at least two sensors are positioned at a proximal end of the handpiece (12), and at least one sensor is located at a distal end of the handpiece (12).

Example 4: The surgical system (10) of any one of examples 1 to 3, wherein the controller (38) is configured to perform the sensor diagnostic routine by processing the pressure data of the at least three sensors (52a, 52b, 54, 56a, 56b, 58) in accordance with a majority rule.

Example 5: The surgical system (10) of any one of examples 1 to 4, wherein the controller (38) is configured to perform the sensor diagnostic routine by processing the pressure data of the at least three sensors (52a, 52b, 54, 56a, 56b, 58) in accordance with respective location of the sensors and Bernoulli's principle of hydrodynamics, and determining if pressure data corresponds with expected pressure variation.

Example 6: The surgical system (10) of any one of examples 1 to 5, wherein the controller (38) is configured to perform a sensor diagnostic routine by processing the pressure data of the at least three sensors (52a, 52b, 54, 56a, 56b, 58), comparing pressure data between the at least three sensors(52a, 52b, 54, 56a, 56b, 58), determine a level of variation in pressure data between the at least three sensors (52a, 52b, 54, 56a, 56b, 58), analyze variation in pressure data between the at least three sensors (52a, 52b, 54, 56a, 56b, 58) in accordance with relative location between the at least three sensors (52a, 52b, 54, 56a, 56b, 58) and the Bernoulli’s principle of hydrodynamics, and determine a malfunction status of one or more pressure sensors (52a, 52b, 54, 56a, 56b, 58) based on the variation in pressure data between the at least three sensors (52a, 52b, 54, 56a, 56b, 58).

Example 7: The surgical system (10) of any one of examples 1 to 6, wherein the pressure sensor assembly (51) is positioned for sensing pressure along at least one of: an irrigation channel (34a) configured to provide irrigation fluid toward the eye (20) of the patient; an aspiration channel (46a) configured for aspirating fluid and tissue material from the eye (20) of the patient; irrigation line transmitting irrigation fluid between the pumping module (24) and the handpiece (12), and aspiration line transmission aspirated fluid and tissue between the handpiece (12) and the pumping module (26).

Example 8: The surgical system (10) of any one of examples 1 to 7, wherein the handpiece (12) further comprises a piezoelectric vibration element (22), the distal end comprises a needle (16) and a sleeve (17), and is configured to be inserted into the eye (20) and to be vibrated by the piezoelectric vibration element (22) to emulsify a lens (18) of the eye (20).

Example 9: The surgical system (10) of any one of examples 1 to 8, wherein the pumping module (24, 26) comprises: an irrigation module (24) configured to supply a flow of irrigation fluid into the eye (20), the irrigation module is configured to be coupled with an irrigation channel (34a) of the handpiece (12) via an irrigation line so as to enable flow of irrigation fluid therethrough; and an aspiration module (26) configured for aspirating eye fluid, the aspiration module (26) being configured to be coupled with an aspiration channel (46a) of the handpiece (12) via an aspiration line so as to enable flow of the eye fluid therethrough.

Example 10: The surgical system (10) of any one of examples 1 to 9, configured as a phacoemulsification system.

Example 11: A method for use in operating a surgical system (10), the method comprising: providing a surgical system (4005) comprising a handpiece (12), a pressure sensor assembly (51), and at least one pumping module (24, 26), wherein the pressure sensor assembly comprises at least three sensors (52a, 52b, 54, 56a, 56b, 58) positioned at two or more locations along a path of fluid flow between the at least one pumping module (24, 26) and a distal end of the handpiece (12), and wherein the at least one pumping module (24, 26) is configured for transmitting and/or evacuating fluid via the handpiece (12); obtaining pressure reading data from the at least three sensors (4030); and performing a sensor diagnostic routine (4040), wherein the sensor diagnostic routine (4040) comprises processing the pressure data from the at least three sensors in accordance with data on rotation rate of one or more pumps of the at least one pumping module (4050), determining correlation between pressure reading data from the at least three sensors and rotation rate of the one or more pumps (4060), and generating an output signal indicative of operation status of the at least three sensors (4070).

Example 12: The method of example 11, wherein the operation status is determined as being functioning or malfunction.

Example 13: The method of any one of examples 11 to 12, wherein the sensor diagnostic routine (4040) comprises processing the pressure data of the at least three sensors, comparing pressure data between the at least three sensors, determining a level of variation in pressure data between the at least three sensors, analyzing variation in pressure data between the at least three sensors in accordance with relative location between the at least three sensors and the Bernoulli’s principle of hydrodynamics, and determining a malfunction status of one or more pressure sensors based on the variation in pressure data between the at least three sensors.

Example 14: A surgical system (10) comprising: a handpiece (12) having a distal end configured to be inserted into an eye (20) of a patient; a pumping module (24, 26) configured for transmitting and/or evacuating fluid via the handpiece (12); a pressure sensor assembly (51) positioned along a fluid flow path between the pumping module (24, 26) and the distal end of the handpiece (12) and configured for providing data on fluid pressure along the fluid flow path; and a controller (38) comprising one or more processors configured for operating the pumping module (24, 26); wherein the pressure sensor assembly (51) comprises at least three sensors (52a, 52b, 54, 56a, 56b, 58) positioned at two or more locations along the fluid flow path between the pumping module (24, 26) and the distal end; and wherein the controller (38) is configured to perform a sensor diagnostic routine by obtaining pressure data from the at least three sensors (52a, 52b, 54, 56a, 56b, 58), processing the pressure data from the at least three sensors (52a, 52b, 54, 56a, 56b, 58) in accordance with a rotation rate of the pumping module (24, 26), and determining malfunction status of the at least three sensors (52a, 52b, 54, 56a, 56b, 58).

Example 15: The surgical system (10) of example 14, wherein the sensor arrangement (51) comprises one or more sensors (52a, 52b, 54, 56a, 56b, 58) located within the handpiece (12).

Example 16: The surgical system (10) of example 14, wherein the three or more sensors (52a, 52b, 54, 56a, 56b, 58) are positioned in at least one of: at least two sensors are positioned at a distal end of the handpiece (12), and at least one sensor is located at a proximal end of the handpiece (12); or at least two sensors are positioned at a proximal end of the handpiece (12), and at least one sensor is located at a distal end of the handpiece (12).

Example 17: The surgical system (10) of examples 14, wherein the controller (38) is configured to perform the sensor diagnostic routine by processing the pressure data of the at least three sensors (52a, 52b, 54, 56a, 56b, 58) in accordance with one or more of: a majority rule, respective location of the sensors and Bernoulli's principle of hydrodynamics, and determining if pressure data corresponds with expected pressure variation, and a rotation rate of the pumping module (24, 26).

Example 18: The surgical system (10) of any one of examples 14 to 17, wherein the controller (38) is configured to perform a sensor diagnostic routine by processing the pressure data of the at least three sensors (52a, 52b, 54, 56a, 56b, 58), comparing pressure data between the at least three sensors(52a, 52b, 54, 56a, 56b, 58), determine a level of variation in pressure data between the at least three sensors (52a, 52b, 54, 56a, 56b, 58), analyze variation in pressure data between the at least three sensors (52a, 52b, 54, 56a, 56b, 58) in accordance with relative location between the at least three sensors (52a, 52b, 54, 56a, 56b, 58) and the Bernoulli’s principle of hydrodynamics, and determine a malfunction status of one or more pressure sensors (52a, 52b, 54, 56a, 56b, 58) based on the variation in pressure data between the at least three sensors (52a, 52b, 54, 56a, 56b, 58).

Example 19: The surgical system (10) of any one of examples 14 to 18, wherein the pressure sensor assembly (51) is positioned for sensing pressure along at least one of: an irrigation channel (34a) configured to provide irrigation fluid toward the eye (20) of the patient; an aspiration channel (46a) configured for aspirating fluid and tissue material from the eye (20) of the patient; irrigation line transmitting irrigation fluid between the pumping module (24) and the handpiece (12), and aspiration line transmission aspirated fluid and tissue between the handpiece (12) and the pumping module (26).

Example 20: The surgical system (10) of any one of examples 14 to 18, wherein the handpiece (12) further comprises a piezoelectric vibration element (22), the distal end comprises a needle (16) and a sleeve (17), and is configured to be inserted into the eye (20) and to be vibrated by the piezoelectric vibration element (22) to emulsify a lens (18) of the eye (20), and wherein the pumping module (24, 26) comprises: an irrigation module (24) configured to supply a flow of irrigation fluid into the eye (20), the irrigation module is configured to be coupled with an irrigation channel (34a) of the handpiece (12) via an irrigation line so as to enable flow of irrigation fluid therethrough; and an aspiration module (26) configured for aspirating eye fluid, the aspiration module (26) being configured to be coupled with an aspiration channel (46a) of the handpiece (12) via an aspiration line so as to enable flow of the eye fluid therethrough.

Example 21: The surgical system (10) of any one of examples 14 to 18, configured as a phacoemulsification system.

Example 22: A method for use in operating a surgical system (10), the method comprising: providing a surgical system (4005) comprising a handpiece (12), a pressure sensor assembly (51), and at least one pumping module (24, 26), wherein the pressure sensor assembly comprises at least three sensors (52a, 52b, 54, 56a, 56b, 58) positioned at two or more locations along a path of fluid flow between the at least one pumping module (24, 26) and a distal end of the handpiece (12), and wherein the at least one pumping module (24, 26) is configured for transmitting and/or evacuating fluid via the handpiece (12); obtaining pressure reading data from the at least three sensors (4030); and performing a sensor diagnostic routine (4040), wherein the sensor diagnostic routine (4040) comprises processing the pressure data from the at least three sensors in accordance with data on rotation rate of one or more pumps of the at least one pumping module (4050), determining correlation between pressure reading data from the at least three sensors and rotation rate of the one or more pumps (4060), and generating an output signal indicative of operation status of the at least three sensors (4070)..

Example 23: The method of example 22, wherein the sensor diagnostic routine further comprises processing the pressure data of the at least three sensors, comparing pressure data between the at least three sensors, determining a level of variation in pressure data between the at least three sensors, analyzing variation in pressure data between the at least three sensors in accordance with relative location between the at least three sensors and the Bernoulli’s principle of hydrodynamics, and determining a malfunction status of one or more pressure sensors based on the variation in pressure data between the at least three sensors.

The present disclosure thus provides for a phacoemulsification system and a respective method enabling monitoring of the operation status of one or more pressure sensors. The present technique provides monitoring of sensor conditions, enabling to generate an alert when one or more sensors malfunction, while allowing a physician to proceed with a surgical operation, and reducing risk of damage to the patient’s eye. The technique of the present disclosure utilizes a pressure sensor assembly comprising three or more sensors and processing of correlation of pressure readings between the sensors, and may also utilize correlation between pressure readings of the sensors and rotation rate of the respective pump.

It is to be noted that the various features described in the various examples can be combined according to all possible technical combinations.

It is to be understood that the invention is not limited in its application to the details set forth in the description contained herein or illustrated in the drawings. The invention is capable of other examples and of being practiced and carried out in various ways. Hence, it is to be understood that the phraseology and terminology employed herein are for the purpose of description and should not be regarded as limiting. As such, those skilled in the art will appreciate that the conception upon which this disclosure is based can readily be utilized as a basis for designing other structures, methods, and systems for carrying out the several purposes of the presently disclosed subject matter.

Those skilled in the art will readily appreciate that various modifications and changes can be applied to the examples of the invention as hereinbefore described without departing from its scope, defined in and by the appended claims.

1. A surgical system comprising:
(a) a handpiece having a distal end configured to be inserted into an eye of a patient;
(b) a pumping module configured for transmitting and/or evacuating fluid via the handpiece;
(c) a pressure sensor assembly positioned along a fluid flow path between the pumping module and the distal end of the handpiece and configured for providing data on fluid pressure along the fluid flow path; and
(d) a controller comprising one or more processors configured for operating the pumping module;
wherein the pressure sensor assembly comprises at least three sensors positioned at two or more locations along the fluid flow path between the pumping module and the distal end; and
wherein the controller is configured to perform a sensor diagnostic routine by obtaining pressure data from the at least three sensors, processing the pressure data from the at least three sensors in accordance with a rotation rate of the pumping module, and determining malfunction status of the at least three sensors.

2. The surgical system of claim 1, wherein the sensor arrangement comprises one or more sensors located within the handpiece.

3. The surgical system of claim 1, wherein the three or more sensors are positioned such that at least two sensors are positioned at a distal end of the handpiece, and at least one sensor is located at a proximal end of the handpiece.

4. The surgical system of claim 1, wherein the three or more sensors are positioned such that at least two sensors are positioned at a proximal end of the handpiece, and at least one sensor is located at a distal end of the handpiece.

5. The surgical system of claim 1, wherein the controller is configured to perform the sensor diagnostic routine by processing the pressure data of the at least three sensors in accordance with a majority rule.

6. The surgical system of claim 1, wherein the controller is configured to perform the sensor diagnostic routine by processing the pressure data of the at least three sensors in accordance with respective location of the sensors and Bernoulli's principle of hydrodynamics, and determining if pressure data corresponds with expected pressure variation.

7. The surgical system of claim 1, wherein the controller is configured to perform a sensor diagnostic routine by processing the pressure data of the at least three sensors, comparing pressure data between the at least three sensors, determine a level of variation in pressure data between the at least three sensors, analyze variation in pressure data between the at least three sensors in accordance with relative location between the at least three sensors and the Bernoulli’s principle of hydrodynamics, and determine a malfunction status of one or more pressure sensors based on the variation in pressure data between the at least three sensors.

8. The surgical system of claim 1, wherein the pressure sensor assembly is positioned for sensing pressure along an irrigation channel, wherein the irrigation channel is configured to provide irrigation fluid toward the eye of the patient.

9. The surgical system of claim 1, wherein the pressure sensor assembly is positioned for sensing pressure along an aspiration channel, wherein the aspiration channel is configured for aspirating fluid and tissue material from the eye of the patient.

10. The surgical system of claim 1, comprising a first pressure sensor assembly comprising at least three sensors positioned for sensing pressure along an aspiration channel or an aspiration line and a second pressure sensor assembly comprising at least three sensors positioned for sensing pressure along an irrigation channel or an irrigation line.

11. The surgical system of claim 1, wherein the handpiece further comprises a piezoelectric vibration element, the distal end comprises a needle and a sleeve, and is configured to be inserted into the eye and to be vibrated by the piezoelectric vibration element to emulsify a lens of the eye.

12. The surgical system of claim 1, wherein the pumping module comprises: an irrigation module configured to supply a flow of irrigation fluid into the eye, the irrigation module is configured to be coupled with an irrigation channel of the handpiece via an irrigation line so as to enable flow of irrigation fluid therethrough; and an aspiration module configured for aspirating eye fluid, the aspiration module being configured to be coupled with an aspiration channel of the handpiece via an aspiration line so as to enable flow of the eye fluid therethrough.

13. The surgical system of claim 1, configured as a phacoemulsification system.

14. A phacoemulsification system comprising:
(a) a handpiece comprising a piezoelectric element and a distal end comprising a needle and a sleeve, wherein the distal end is configured to be inserted into an eye and to be vibrated by the piezoelectric element to emulsify a lens of the eye;
(b) an irrigation module configured to supply a flow of irrigation fluid into the eye, the irrigation module being configured to be coupled with an irrigation channel of the handpiece via an irrigation line so as to enable flow of irrigation fluid therethrough;
(c) an aspiration module configured for aspirating eye fluid; the aspiration module is configured to be coupled with an aspiration channel of the handpiece via an aspiration line so as to enable flow of the eye fluid therethrough;
(d) a pressure sensor assembly coupled with at least one of the irrigation channel, the irrigation line, the aspiration channel, and the aspiration line, and configured to monitor at least one of an irrigation pressure in the irrigation channel, an irrigation pressure in the irrigation line, an aspiration pressure in the aspiration channel, and an aspiration pressure in the aspiration line; and
(e) a controller comprising one or more processors and configured for operating the irrigation module and aspiration module; wherein the pressure sensor assembly comprises at least three sensors positioned at two or more locations along a path of fluid flow between the pumping module and the distal end; and
wherein the pressure sensor assembly comprises at least three sensors positioned at two or more locations along a path of fluid flow between the pumping module and the distal end; and
wherein the controller is further configured for performing a sensor diagnostic routine comprising obtaining pressure data from the at least three sensors of the pressure sensor assembly, processing the pressure data from the at least three sensors in accordance with a rotation rate of the irrigation module or the aspiration module, and determine malfunction status of one or more of the at least three sensors.

15. A method for use in operating a surgical system, the method comprising:
(a) providing a surgical system comprising a handpiece, a pressure sensor assembly, and at least one pumping module, wherein the pressure sensor assembly comprises at least three sensors positioned at two or more locations along a path of fluid flow between the at least one pumping module and a distal end of the handpiece, and wherein the at least one pumping module is configured for transmitting and/or evacuating fluid via the handpiece;
(b) obtaining pressure reading data from the at least three sensors; and
(c) performing a sensor diagnostic routine, wherein the sensor diagnostic routine comprises processing the pressure data from the at least three sensors in accordance with data on rotation rate of one or more pumps of the at least one pumping module, determining correlation between pressure reading data from the at least three sensors and rotation rate of the one or more pumps, and generating an output signal indicative of operation status of the at least three sensors.

16. The method of claim 15, wherein the operation status is determined as being functioning or malfunction.

17. The method of claim 15, wherein the sensor diagnostic routine further comprises processing the pressure data of the at least three sensors, comparing pressure data between the at least three sensors, determining a level of variation in pressure data between the at least three sensors, analyzing variation in pressure data between the at least three sensors in accordance with relative location between the at least three sensors and the Bernoulli’s principle of hydrodynamics, and determining a malfunction status of one or more pressure sensors based on the variation in pressure data between the at least three sensors.
